SEQUENCE LISTING (11 sequences)

SEQ ID NO: 1 - DNA, 25 bp, Artificial Sequence
  Description of Artificial Sequence primer
ttctccccagacgccgaggatggcc

SEQ ID NO: 2 - DNA, 24 bp, Artificial Sequence
  Description of Artificial Sequence primer
tgttggtcccaattgtctcccctc

SEQ ID NO: 3 - DNA, 18 bp, Artificial Sequence
  Description of Artificial Sequence primer
cactccatgaggtatttc

SEQ ID NO: 4 - DNA, 15 bp, Artificial Sequence
  Description of Artificial Sequence primer
tctggttgtagtagc

SEQ ID NO: 5 - DNA, 15 bp, Artificial Sequence
  Description of Artificial Sequence primer
ggccagggtctcaca

SEQ ID NO: 6 - DNA, 15 bp, Artificial Sequence
  Description of Artificial Sequence primer
caattgtctcccctc

SEQ ID NO: 7 - DNA, 24 bp, Artificial Sequence
  Description of Artificial Sequence primer
acccacccggactcagaatctcct

SEQ ID NO: 8 - DNA, 23 bp, Artificial Sequence
  Description of Artificial Sequence primer
ggaggccatccccggcgacctat

SEQ ID NO: 9 - DNA, 23 bp, Artificial Sequence
  Description of Artificial Sequence primer
ggaggccatccccggcgatctat

SEQ ID NO: 10 - DNA, 14 bp, Artificial Sequence
  Description of Artificial Sequence primer
ccccggcgacctat

SEQ ID NO: 11 - DNA, 15 bp, Artificial Sequence
  Description of Artificial Sequence primer
ggaaggctccccact

(19) United States
(12) Patent Application Publication
(10) Pub. No.: US 2004/0175746 A1
(43) Pub. Date: Sep. 9, 2004

(54) Method for HLA-typing

(76) Inventors: Masamitsu Shikata, Kyoto-shi (JP); Tomoko Inagaki, Osaka (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/785,966

(22) Filed: Feb. 26, 2004

(30) Foreign Application Priority Data
Mar. 4, 2003 (JP) PAT.2003-57287

(51) Int. Cl. C12Q 1/68

(52) U.S. Cl. 435/6

(57) ABSTRACT

The present invention provides a HLA-typing technique that uses samples that are safe and convenient for storage and transportation and therefore can significantly reduce the cost associated with the analysis and ensure high accuracy of the analysis as well as high processing efficiency. A method for HLA typing, comprising the steps of: directly amplifying DNA from a biological sample; determining a base sequence of DNA in the biological sample; and determining HLA type on the basis of the base sequence. Preferably, the DNA amplification reaction is Polymerase Chain Reaction, the biological sample is a paper-spotted blood sample obtained by spotting blood on filter paper and drying the blood.

### BACKGROUND OF THE INVENITION

1. Field of the Invention

The present invention relates to a technique for typing human leukocyte antigens (HLAs) used for medical diagnosis.

2. Disclosure of the Related Art

Human leukocyte antigens (HLAs) are human major histocompatibility complex (MHC) molecules and are found on the surface of all cells. Different types of HLA molecules are expressed in different individuals and bind to different peptide antigens. This gives rise to the differences in immune response among individuals. For this reason, determination of the types of HLA-encoding genes in the HLA region (HLA typing) serves as a key technology in determining the compatibility between a donor and a recipient in organ transplantation and evaluating the susceptibility of an individual to particular diseases.

Two different approaches are known for HLA typing: serological typing and DNA typing. In serological typing, which has been widely used in HLA-typing, HLA types are identified based on the antigen/antibody interactions between anti-HLA monoclonal antibodies and lymphocytes. However, this approach has disadvantages that the accuracy of the typing is relatively low and that it only allows determination of limited types of HLA.

The DNA typing on the other hand is a technique that directly determines the types of the HLA genes on chromosome 6 (e.g., RFLP). With reference to Japanese National Publication No. 2002-503497 for example, to perform DNA typing, DNA is previously separated/purified from biological samples and is used as a template in a polymerase chain reaction (PCR) along with locus-specific primers to obtain a template for sequencing.

### SUMMARY OF THE INVENTION

Problems of conventional approach include the difficulties in storing and transporting biological samples. In particular, in the case that the biological sample is a blood sample, the blood sample is associated with the risk of transmitting pathogenic microbes and can pose a potential health risk to those who are exposed to blood samples during testing of the samples. Blood and other liquid samples are also susceptible to the risk of contamination of the samples themselves, as in the case of cross-contamination among the samples. Furthermore, the separation/purification of biological samples requires significant amounts of labor and costs. Though the recent development of a variety of kits has made the DNA separation/purification relatively simple, the separation/purification of DNA is still a tedious process involving multiple steps. Thus, when analysis of multiple analytes is required, not only is the safety of the samples put at risk, but the analysis becomes costly.

It is thus an objective of the present invention to provide a HLA-typing technique that uses samples that are safe and convenient for storage and transportation, and therefore can significantly reduce the cost associated with the analysis and ensure high accuracy as well as high processing efficiency.

Over the course of the inventors' studies, the present inventors have found that the above-described objective can be achieved by directly amplifying DNA from a biological sample, sequencing the amplified DNA, and determining HLA types on the basis of the base sequence information obtained. This finding ultimately led the present inventors to devise the present invention.

The present invention comprises following inventions:

(1) A method for HLA typing, comprising the steps of:

directly amplifying DNA from a biological sample;

determining a base sequence of DNA in the biological sample; and

determining HLA type on the basis of the base sequence.

(2) The method of above (1), wherein the DNA amplification reaction is Polymerase Chain Reaction.

(3) The method of above (1) or (2), wherein the biological sample is a blood sample or an oral mucosa sample.

(4) The method of above (3), wherein the blood sample is a whole blood.

(5) The method of above (3) or (4), wherein the blood sample is a dried blood.

(6) The method of any of above (3) to (5), wherein the blood sample is a paper-spotted blood sample obtained by spotting blood on filter paper and drying the blood.

According to the present invention, a HLA-typing technique, that uses samples that are safe and convenient for storage and transportation and therefore can significantly reduce the cost associated with the analysis and ensure high accuracy of the analysis as well as high processing efficiency, are provided. The present invention also allows the typing of all HLA loci by selecting a buffer suitable for the direct PCR.

### BRIEF DESCRIPTION OF THE DRAWINGS

FIG. 1 shows the results of electrophoresis performed on PCR products directly amplified from paper-spotted blood samples.

FIG. 2 shows the results of electrophoresis preformed on PCR products directly amplified from oral mucosa samples.

### DETAILED DESCRIPTION OF THE INVENTION

The present invention provides a novel HLA-typing method, characterized in that it involves DNA amplification directly from biological samples. As used herein, the term “directly” means that the separation/purification of DNA containing in the biological samples is not required prior to the amplification of DNA. According to the HLA-typing method of the present invention, DNA is directly amplified from biological samples, the amplified DNA fragments are sequenced to determine the base sequences of DNA in the biological samples, and HLA types are determined on the basis of the base sequences. Typically, HLA types are determined for the each locus of HLA-A, -B, and -DR.

In the present invention, the biological sample for HLA typing is not particularly limited, and a blood sample and an oral mucosa sample are preferably used in that they are convenient to obtain samples.

The blood sample for use in the present invention may be whole blood. Purified DNA or EDTA-treated DNA may also be used. The blood samples may be provided in the form of any of fresh blood, stored blood, or dried blood, provided that they contain DNA. The stored blood may be either cold-storage blood or frozen blood. In the present invention, amplified nucleic acid products can be obtained in a stable manner from frozen blood that has been frozen and thawed repeatedly. The dried blood may be in the form of blood spotted on filter paper and then dried (paper-spotted blood). The use of the paper-spotted samples is preferred in the present invention as they are convenient for storage and transportation and are highly safe. Since blood samples spotted on filter paper no longer takes a liquid form, the samples are less susceptible to the risk of spilling and spattering. Such blood samples therefore are safe and are less likely to be contaminated.

According to present invention, the DNA present in the blood samples described above is amplified without being subjected to a separation/purification process. In the present invention, specific reaction mixture, such as those described later, are used in the amplification reaction to allow the blood samples to be directly used in DNA amplification without any pretreatment. As a result, the amounts of labor and costs associated with pretreatment can be significantly decreased. In one example in which paper-spotted blood samples are used, a small amount of blood is spotted on filter paper and is dried. An area of the blood spot is then punched out and is directly subjected to the amplification reaction.

A preferred technique for the DNA amplification for use in the present invention is PCR. A typical PCR reaction mixture contains a buffer, a set of primers, dNTPs (Deoxynucleoside triphosphates), and DNA polymerase.

Without being subjected to pretreatment such as separation or purification, the samples contain compounds that inhibit DNA amplification. Such inhibitors of DNA amplification include positively charged compounds, such as certain types of proteins, and negatively charged compounds, such as certain types of sugars and pigments, that are present in biological fluids. Proteins and other positively charged compounds inhibit amplification reaction by binding to DNA, whereas sugars, pigments and other negatively charged compounds inhibit amplification by binding to DNA polymerase. For this reason, a buffer capable of suppressing the activity of these inhibitors of DNA amplification is used in the present invention.

Examples of such buffers are Ampdirect®-A and Ampdirect®-G/C (each manufactured from Shimadzu Corporation). When necessary, Amp Addition-1 or Amp Addition-4, a reagent to increase the efficiency of the amplification (each manufactured by Shimadzu Corporation and collectively referred to as Amp Addition, hereinafter), may be added to Ampdirect®-A or Ampdirect®-G/C. Ampdirect®-G/C is particularly preferred for use in the present invention. More preferably, Amp Addition-4 is added to Ampdirect®-G/C. The Ampdirect®-A and Ampdirect®-G/C each act to neutralize the above-described inhibitors present in the DNA amplification mixture, thereby allowing unpurified samples to be used in PCR. This allows the direct amplification of nucleic acids from biological samples. Other buffers having the same activities include Ampdirect®-B and Ampdirect®-D, while other reagents of the Amp Addition series optionally added to the buffer include Amp Addition-2 and Amp Addition-3.

The buffers for use in the present invention are not limited to those described above and a buffer having suitable composition may be properly selected depending on the types of loci.

Preferred primers for use in the present invention include amplification primer sets specific to the HLA region. Preferred DNA polymerases for use in the present invention include Taq DNA polymerase. Taq DNA polymerases such as TaKaRaZ-Taq™ and ExTaq (each available from Takara Shuzo Co., Ltd.) are particularly preferred.

The above-described components of the PCR reaction mixture of the present invention may be contained in the following amounts (with respect to the entire volume of the PCR mixture): 10 to 40 vol %, for example 20 vol %, of the buffer; 0.1 to 2 μM, for example 0.5 μM, of each primer; 100 to 300 μM, for example 200 μM, of each dNTP; and 0.5 to 1.25 U/μl, for example 1.25 U/μl, of DNA polymerase. In the case of adding Amp Addition to Ampdirect®-A or Ampdirect®-G/C to make the buffer, as much as approximately 150 vol %, for example 100 vol %, of Amp Addition may be added with respect to the amount of Ampdirect®-A or Ampdirect®-G/C while it may be added in any amount with no specific upper limit. The blood sample, for example, may be used in an amount of 0.5 μl to 2 μl and the PCR mixture may be used in an amount of 10 to 200 μl, preferably in an amount of 20 to 100 μl, with respect to 1 μl of the blood sample. These conditions, as well as the conditions for the PCR reaction, may be properly determined by those skilled in the art.

The amplified DNA is preferably purified. The purification may be carried out using exonuclease and alkaline phosphatase. These enzymes act to inactivate unreacted primer nucleotides and thus facilitate the purification of the amplified DNA products.

Once purified, the sequences of the DNA fragments are determined. Accordingly, the method of the present invention, which is essentially a DNA-typing method and allows determination of the base sequence of the HLA region, achieves more accurate HLA typing as compared to conventional serological HLA-typing techniques.

The base sequences may be determined by Sanger method, Maxam-Gilbert method and other proper sequencing techniques. In Sanger method, for example, amixture of partially replicated DNA fragments having different lengths is prepared by an ordinary reaction process. The partially replicated DNA fragments obtained may be subjected to sequencing on a capillary- or a slab gel-type DNA sequencer, each relying upon the principle of electrophoresis. In the present invention, capillary DNA sequencers RISA-384 (Shimadzu Corporation) and ABI-3730x1 (Applied Biosystems) are preferably used. The obtained sequence data are analyzed by a computer program in such a manner that the double peaks reflecting the presence of heterozygous conjugates are automatically detected and the consensus sequences are determined. HLA types of the base sequences are then automatically determined by searching the HLA-type database.

As described above, the present invention has led to a significant decrease in the amounts of labor and cost associated with DNA analysis without sacrificing the high accuracy of DNA typing. Thus, the invention allows more effective processing of a larger number of samples than is possible by the use of conventional approaches.

### EXAMPLES

The present invention will now be described with reference to examples, which are provided by way of example only and are not intended to limit the scope of the invention in any way.

### Example 1

Small amounts of blood (approx. 1 μl) were collected from forty subjects by pricking their fingertip with a needle and were individually spotted on paper filter. An area of each spot, 2 mm in diameter, was punched out with a punch and was placed in each well of 96-well PCR plate. 20 μl of a PCR mixture, the composition of which is shown below, were added to the blood-spotted filter paper in each well of the plate and the plate was sealed to prevent evaporation.

(PCR Mixture in 20 μl))

| Primers | | |
|---|---|---|
| TTCTCCCCAGACGCCGAGGATGGCC | (SEQ ID NO:1) | 0.5 μM |
| TGTTGGTCCCAATTGTCTCCCCTC | (SEQ ID NO:2) | 0.5 μM |
| Ampdirect ® -G/C (Shimadzu Corporation) | | 4 μl |
| dATP, dCTP, dGTP, dTTP | | 200 μM each |
| ExTaq (Takara Shuzo Co., Ltd.) | | 0.5 U |

A PCR reaction was carried out on a thermal cycler (GeneAmp 9700, Applied Biosystems) with the following settings: preheating at 96° C. for 3 min, followed by 40 cycles of 96° C. for 30 sec, 63° C. for 1 min and 72° C. for 3 min, with the final polymerization at 72° C. for 5 min.

To 10 μl of each reaction mixture collected, 1 μl of a mixture of exonuclease and alkaline phosphatase (ExoSAP-IT, Amersham) was added for purification.

Using 5 μl of a sequencing reaction mixture shown below, each of the purified PCR products was sequenced with the four primers shown below.

(Sequencing Reaction Mixture (in 5 μl))

| | |
|---|---|
| The purified PCR product | 0.25 μl |
| Primer | 1 μM |
| BigDye Terminator Ver3.1 (Applied Biosystems) | 1 μl |

| (Primer) | | |
|---|---|---|
| 1. | CACTCCATGAGGTATTTC | (SEQ ID NO: 3) |
| 2. | TCTGGTTGTAGTAGC | (SEQ ID NO: 4) |
| 3. | GGCCAGGGTCTCACA | (SEQ ID NO: 5) |
| 4. | CAATTGTCTCCCCTC | (SEQ ID NO: 6) |

The partially replicated DNA fragments obtained in the sequencing reaction were purified by ethanol precipitation, were redissolved in water, and were then sequenced on a capillary DNA sequencer (RISA-384, Shimadzu Corporation). The double peaks reflecting the presence of heterozygous conjugates were detected to determine the consensus sequences. Then, the HLA type data available on http://square.umin.ac.jp/JSHI/mhc.html was searched for matches with the consensus sequences and the hits were listed.

A comparison between the HLA types determined by the above-described typing method and the HLA types determined by the conventional serological typing method showed a complete match for all of the forty samples tested.

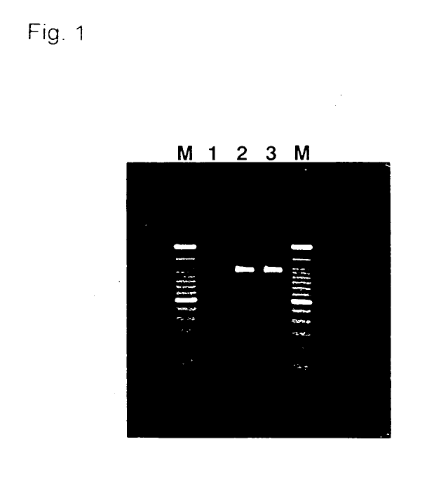

Whether the DNA was directly PCR-amplified from the paper-spotted blood was verified by subjecting some of the amplification products to electrophoresis. The results of electrophoresis are shown in FIG. 1. Referring to FIG. 1, Lane 1 shows the results obtained by replacing 4 μl of the buffer Ampdirect®-G/C used in PCR in Example 1 for 2 μl of a PCR buffer contained with ExTaq (Takara Shuzo Co., Ltd.). Lane 2 indicates the results of PCR in Example 1. Lane 3 shows the results obtained by replacing 4 μl of the buffer Ampdirect®-G/C used in PCR in Example 1 for 8 μl of a mixture of Ampdirect®-G/C and Amp Addition-4 (1:1 by volume). As shown by the results in FIG. 1, no DNA amplification was detected for the conventional PCR buffer (Lane 1), whereas significant DNA amplification was observed when Ampdirect®-G/C or the mixture of Ampdirect®-G/C and Amp Addition-4 was used (Lanes 2 and 3, respectively).

### Example 2

Five subjects were asked to wash their mouths with small amounts of water. The subjects were then asked to brush their buccal surfaces with a commercial toothbrush 10 times for each cheek (20 times for both cheeks). Subsequently, the toothbrushes were each placed in a centrifuge tube containing a 10 ml 1 wt % aqueous solution of NaCl and the tubes were shaken several times to suspend the collected buccal mucosa in the solution, to obtain a sample suspension.

To a test tube containing 45 μl of a PCR reaction mixture, the composition of which is shown below, a 5 μl portion of the obtained sample suspension was added.

(PCR mixture in 50 μl))

| Primers | | |
|---|---|---|
| Forward Primer | | |
| ACCCACCCGGACTCAGAATCTCCT | (SEQ ID NO: 7) | 0.5 μM |
| Reverse Primer (used as a mixed primer of following two primers) | | |
| GGAGGCCATCCCCGGCGACCTAT | (SEQ ID NO: 8) | 0.25 μM |
| GGAGGCCATCCCCGGCGATCTAT | (SEQ ID NO: 9) | 0.25 μM |
| Ampdirect ® -G/C (Shimadzu Corporation) | | 10 μl |
| Amp Addition-4 (Shimadzu Corporation) | | 10 μl |
| dATP, dCTP, dGTP, dTTP | | 200 μM each |
| Z-Taq (Takara Shuzo Co., Ltd.) | | 1.25 U |

A PCR reaction was carried out on a thermal cycler (GeneAmp 9700, Applied Biosystems) with the following settings: pretreatment at 80° C. for 15 min, followed by preheating at 96° C. for 3 min, 40 cycles of 96° C. for 30 sec, 63° C. for 1 min and 72° C. for 3 min, with the final polymerization at 72° C. for 5 min.

To 10 μl of each reaction mixture collected, 1 μl of a mixture of exonuclease and alkaline phosphatase (ExoSAP-IT, Amersham) was added for purification.

Using 5 μl of a sequencing reaction mixture shown below, each of the purified PCR products was sequenced with the four primers shown below.

(Sequencing Reaction Mixture (in 5 μl))

| | |
|---|---|
| The purified PCR product | 0.25 μl |
| Primer | 1 μM |
| BigDye Terminator Ver3.1 (Applied Biosystems) | 1 μl |

| (Primer) | | |
|---|---|---|
| 1. | CACTCCATGAGGTATTTC | (SEQ ID NO: 3) |
| 2. | TCTGGTTGTAGTAGC | (SEQ ID NO: 4) |
| 3. | GGCCAGGGTCTCACA | (SEQ ID NO: 5) |
| 4. | a mixed primer of following two primers | |
| | CCCCGGCGACCTAT | (SEQ ID NO: 10) |
| | GGAAGGCTCCCCACT | (SEQ ID NO: 11) |

The partially replicated DNA fragments obtained in the sequencing reaction were purified by ethanol precipitation, were redissolved in water, and were then sequenced on a capillary DNA sequencer (RISA-384, Shimadzu Corporation). The double peaks reflecting the presence of heterozygous conjugates were detected to determine the consensus sequences. Then, the HLA type data available on http://square.umin.ac.jp/JSHI/mhc.html was searched for matches with the consensus sequences and the hits were listed.

A comparison between the HLA types determined by the above-described typing method and the HLA types determined by the conventional serological typing method showed a complete match for all of the five samples tested.

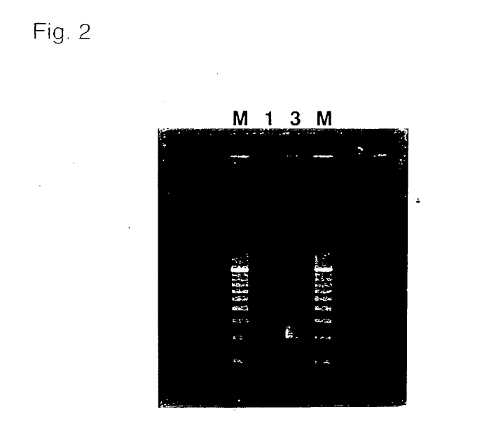

Whether the DNA was directly PCR-amplified from the oral mucosa was verified by subjecting some of the amplification products to electrophoresis. The results of electrophoresis are shown in FIG. 2. Referring to FIG. 2, Lane 1 shows the results obtained by replacing 4 μl of the buffer Ampdirect®-G/C used in PCR in Example 1 for 2 μl of a PCR buffer contained with ExTaq (Takara Shuzo Co., Ltd.). Lane 3 indicates the results of PCR in Example 2. As shown by the results in FIG. 2, no DNA amplification was detected for the conventional PCR buffer (Lane 1), whereas significant DNA amplification was observed when the mixture of Ampdirect®-G/C and Amp Addition-4 was used (Lanes 3).

The above-described Examples shows concrete two modes within the scope of the present invention, however, the present invention can be carried out in various other modes. Therefore, the above-described Examples are merely illustrative in all respects, and must not be construed as being restrictive. Further, the changes that fall within the equivalents of the claims are all within the scope of the present invention.

What is claimed is:

1. A method for HLA typing, comprising the steps of:
directly amplifying DNA from a biological sample;
determining a base sequence of DNA in the biological sample; and
determining HLA type on the basis of the base sequence.

2. The method according to claim 1, wherein the DNA amplification reaction is Polymerase Chain Reaction.

3. The method according to claim 1, wherein the biological sample is a blood sample or an oral mucosa sample.

4. The method according to claim 3, wherein the blood sample is a whole blood.

5. The method according to claim 3, wherein the blood sample is a dried blood.

6. The method according to claim 1, wherein the blood sample is a paper-spotted blood sample obtained by spotting blood on filter paper and drying the blood.
